(19) United States
(12) Patent Application Publication
(10) Pub. No.: US 2012/0252006 A1
(43) Pub. Date: Oct. 4, 2012

(54) Methods and Systems for Multiple Control Validation

(75) Inventors: Elizabeth M. Rohlfs, Hopkington, MA (US); Nicole Evan Castro, Acton, MA (US)

(73) Assignee: LABORATORY CORPORATION OF AMERICA HOLDINGS, Burlington, NC (US)

(21) Appl. No.: 13/426,140

(22) Filed: Mar. 21, 2012

Related U.S. Application Data
(60) Provisional application No. 61/454,723, filed on Mar. 21, 2011.

(51) Int. Cl. G01N 33/53; G01N 33/566; C12Q 1/68; C12M 1/34; G01N 30/00; C12Q 1/70

(52) U.S. Cl. 435/5; 422/69; 435/6.12; 435/7.1; 435/287.1; 435/287.2; 436/501

(57) ABSTRACT

The invention provides methods for validating a multiplex binding assay that results in a reduced number of false invalidations. The invention further provides systems for validating a multiplex binding assay that results in a reduced number of false invalidations. The invention further provides a computer readable medium containing program instructions for validating a multiplex binding assay that results in a reduced number of false invalidations.

### CROSS-REFERENCE TO RELATED APPLICATIONS

The present application claims the benefit of priority to U.S. Provisional Patent Application No. 61/454,723, filed Mar. 21, 2011, which is incorporated by reference in its entirety as though fully set forth herein.

### FIELD OF THE INVENTION

The invention relates to methods for validating a multiplex binding assay that results in a reduced number of false invalidations. The invention further relates to systems for validating a multiplex binding assay that results in a reduced number of false invalidations. The invention further relates to a computer readable medium containing program instructions for validating a multiplex binding assay that results in a reduced number of false invalidations.

### BACKGROUND OF THE INVENTION

Binding assays, such as multiplex hybridization assays, provide a common means for detecting the presence or amount of multiple analytes in a solution, such as a solution derived from a biological sample. Such assays are often used to detect biomarkers or groups of biomarkers. These biomarkers may relate to the diagnosis of a disease, such as cystic fibrosis, identification of individuals who are carriers of a disease mutation, viral infections, gastrointestinal disorders, or any disease or condition that has a recognizable molecular and/or genomic signature. These biomarkers may also be useful for designing treatment regimens that are specific to an individual subject or a class of subjects. In such instances, certain biomarkers or groups of biomarkers may indicate a need for changing the course of a treatment regimen, or can also indicate the likelihood of the effectiveness of a particular treatment program (e.g., in instances where there is a known genotypic basis for resistance to certain drug therapies).

In traditional multiplex binding assays, such as bead-array hybridization assays, the assay is validated by contacting one or more beads with a control solution, where the control solution contains no significant (or detectable) amount of one or more target analytes. If the binding of the control solution to each bead is below a predetermined threshold amount, the assay is considered to be validated. But such validation methods result in an unacceptably high number of false invalidations, and uncompromised assays are needlessly discarded for having failed the validation protocol.

There is no clear cause for these false validations. Generally, if the control solution contains no significant amount an analyte, one would not expect the assay to generate a signal indicative of the analyte's presence. Various causes for these false positives have been suggested, including interference from the presence of non-specific polymerase chain reaction (PCR) products, interference from non-specific allele-specific primer extension (ASPE) products, or interference from unincorporated tags that may fail to be washed out following hybridization. Whatever the reason for the false positives, use of the standard validation protocol can require one to discard a substantial number of assays that are uncompromised and that would otherwise accurately indicate the presence or amount of a target analyte in a test solution. This involves needless waste of materials, including waste of the biological sample from which the test solution may have been derived. In some such instances, additional biological samples may need to be obtained, which places an immense burden on the subject who supplied the sample and on the professionals who collect such samples. Follow-up testing needlessly drives up the cost of obtaining validated test results, and causes delays in sending validated results to physicians, who may be waiting on the information before designing a treatment regimen for the subject, before performing additional testing, or before providing a diagnosis to the patient.

For bead-based hybridization assays, one solution to this problem may include validating the assay on a bead-by-bead basis. But a bead-by-bead validation would be time consuming and increase the complexity of the measurement and reporting of information. And because some applications of the technology may depend on obtaining validated results for a plurality of analytes, such as personalized medicine testing, testing related to diagnosing multi-factorial diseases or conditions, or analyzing a plurality of mutations in a single gene or a plurality of genes, bead-by-bead validation could nonetheless lead to the reporting of incomplete (and therefore unsatisfactory) results to the physician. Thus, in the end, the marginal gains achieved by bead-by-bead validation would be offset by such additional inconvenience.

Therefore, there is a continuing need for improved methods and systems for validating binding assays, such as bead-based hybridization assays, so as to reduce, if not eliminate, the number of false invalidations. The present invention, among its other benefits, addresses that need by providing systems and methods for validating a binding assay, where use of such systems and methods results in a reduced number of false invalidations. To that general end, the invention further provides computer readable media that contain program instructions for validating binding assays so as to reduce the number of false invalidations.

### SUMMARY OF THE INVENTION

In at least one aspect, the invention provides methods for validating a multiplex binding assay, the method comprising: (a) providing a binding assay having two or more sets of binding structures, wherein each set of binding structures comprises at least one target-adapted binding structure that is adapted to couple to a target analyte; (b1) contacting a first set of the two or more sets of binding structures with a first negative control solution; and then (b2) determining a binding signal for the at least one target-adapted binding structure from the first set of two or more binding structures; (c1) contacting a second set of the two or more sets of binding structures with a second negative control solution; and then (c2) determining a binding signal for the at least one target-adapted binding structure from the second set of two or more binding structures; and (d) comparing each determined binding signal, or a representative value thereof, to a predetermined threshold and a predetermined limit to determine whether the binding assay is validated.

In another aspect, the invention provides methods for validating a multiplex binding assay, the method comprising: (a) providing a binding assay having two or more sets of binding structures, wherein each set of binding structures comprises at least one target-adapted binding structure of a first type that is adapted to couple to a first target analyte, and at least one target-adapted binding structure of a second type that is adapted to couple to a second target analyte; (b1) contacting a first set of the two or more sets of binding structures with a first negative control solution; and then (b2) determining a binding signal for the at least one target-adapted binding structure of the first type from the first set of two or more binding structures, and a binding signal for the at least one target-adapted binding structure of the second type from the first set of two or more binding structures; (c1) contacting a second set of the two or more sets of binding structures with a second negative control solution; and then (b2) determining a binding signal for the at least one target-adapted binding structure of the first type from the second set of two or more binding structures, and a binding signal for the at least one target-adapted binding structure of the second type from the second set of two or more binding structures; and (d) comparing each determined binding signal, or a representative value thereof, to a predetermined threshold and a predetermined limit to determine whether the binding assay is validated.

In another aspect, the invention provides systems for validating a multiplex binding assay having two or more sets of binding structures wherein each set of binding structures comprises at least one target-adapted binding structure that is adapted to couple to a target analyte, the system comprising: (a) a station for contacting a first set of the two or more sets of binding structures with a first negative control solution; (b) a station for contacting a second set of the two or more sets of binding structures with a second negative control solution; (c) a station for determining a binding signal for the at least one target-adapted binding structure from the first set of two or more binding structures; (d) a station for determining a binding signal for the at least one target-adapted binding structure from the second set of two or more binding structures; and (e) a station for comparing each determined binding signal, or a representative value thereof, to a predetermined threshold and a predetermined limit to determine whether the binding assay is validated.

In another aspect, the invention provides systems for validating a multiplex binding assay having two or more sets of binding structures, wherein each set of binding structures comprises at least one target-adapted binding structure of a first type that is adapted to couple to a first target analyte, and at least one target-adapted binding structure of a second type that is adapted to couple to a second target analyte, the system comprising: (a) a station for contacting a first set of the two or more sets of binding structures with a first negative control solution; (b) a station for contacting a second set of the two or more sets of binding structures with a second negative control solution; (c) a station for determining a binding signal for the at least one target-adapted binding structure of the first type from the first set of two or more binding structures, and a binding signal for the at least one target-adapted binding structure of the second type from the first set of two or more binding structures; (d) a station for determining a binding signal for the at least one target-adapted binding structure of the first type from the second set of two or more binding structures, and a binding signal for the at least one target-adapted binding structure of the second type from the second set of two or more binding structures; and (e) a station for comparing each determined binding signal, or a representative value thereof, to a predetermined threshold and a predetermined limit to determine whether the binding assay is validated.

In another aspect, the invention provides computer readable media for validating a multiplex binding assay, the computer readable medium comprising: (a) program code for determining a binding signal for a target-adapted binding structure, where the target-adapted binding structure is a binding structure that is adapted to couple to a target analyte and subsequently contacted with a first negative control solution; (b) program code for determining a binding signal for a target-adapted binding structure, where the target-adapted binding structure is a binding structure that is adapted to couple to a target analyte and subsequently contacted with a second negative control solution; (c) program code for comparing each determined binding signal, or a representative value thereof, to a predetermined threshold and a predetermined limit; and (d) program code for determining whether to determine whether the multiplex binding assay is validated.

In another aspect, the invention provides computer readable medium for validating a multiplex binding assay, the computer readable medium comprising: (a) program code for determining a binding signal for a target-adapted binding structure of a first type, where the target-adapted binding structure is a binding structure that is adapted to couple to a first target analyte and subsequently contacted with a first negative control solution; (b) program code for determining a binding signal for a target-adapted binding structure of a second type, where the target-adapted binding structure is a binding structure that is adapted to couple to a second target analyte and subsequently contacted with a first negative control solution; (c) program code for determining a binding signal for a target-adapted binding structure of a first type, where the target-adapted binding structure is a binding structure that is adapted to couple to a first target analyte and subsequently contacted with a second negative control solution; (d) program code for determining a binding signal for a target-adapted binding structure of a second type, where the target-adapted binding structure is a binding structure that is adapted to couple to a second target analyte and subsequently contacted with a second negative control solution; (e) program code for comparing each determined binding signal, or a representative value thereof, to a predetermined threshold and a predetermined limit; and (f) program code for determining whether the multiplex binding assay is validated.

Other aspects of the invention are provided below.

### BRIEF DESCRIPTION OF DRAWINGS

FIGS. 1-5 depict embodiments of the invention. The figures and their accompanying descriptions are provided for illustrative purposes and do not limit the scope of the invention.

FIGS. 1A and 1B show system diagrams depicting exemplary computing devices in exemplary computing environments according to various embodiments.

FIGS. 2A and 2B show block diagrams depicting exemplary computing devices according to various embodiments.

FIG. 3 depicts a flow chart for preparing polynucleotide target analytes from a biological sample, such as a sample from a human subject. The biological sample is received 310, where the biological sample includes buccal cells or peripheral blood lymphocytes. DNA is then extracted 320 from the biological sample. Then, using PCR, regions of particular interest on the DNA are amplified 330. Unused reagents or byproducts of PCR are then degraded 340. Then, allele-specific primer extension is performed on the amplified DNA, during which one may incorporate a biotin moiety to effect binding of a reporter 350.

FIG. 4 depicts a flow chart for analyzing polynucleotide target analytes in a multiplex binding assay. The polynucleotide target analytes (i.e., ASPE-transformed PCR-amplified DNA of a subject) 410 is prepared as described in FIG. 3. A solution containing the polynucleotide target analytes is contacted with a set of target-adapted binding structures (e.g., disposed on beads) to allow for hybridization of the target analytes with the target-adapted binding structures 420. A solution containing a reporter is added 430, which can, for example, bind to the biotin in the target-adapted analyte. Using flow cytometry equipped with a fluorescent detection system, a binding signal is determined for various beads 440. These data may be analyzed 450, and, if the assay is validated, reported to the party requesting the analysis.

FIG. 5 depicts a flow chart for the two-control validation process. Using flow cytometry equipped with a fluorescent detection system, a binding signal is recorded 510 for two sets of binding structures, where the binding structures have been incubated in a negative control solution instead of in a test solution. The type of the binding structure is then determined 520. Data collected from both negative controls is stored and analyzed 530, and a representative binding signal value may be calculated for each binding structure type within each set. Such values are compared to a predetermined threshold and a predetermined limit 540. Using the decision chart shown in Tables 1-3, a validation decision is made 550.

### DETAILED DESCRIPTION

The following description recites various aspects and embodiments of the present invention. No particular embodiment is intended to define the scope of the invention. Rather, the embodiments merely provide non-limiting examples various methods and systems that are at least included within the scope of the invention. The description is to be read from the perspective of one of ordinary skill in the art; therefore, information well known to the skilled artisan is not necessarily included.

### Definition and Abbreviations

The following terms, unless otherwise indicated, shall be understood to have the following meanings

As used herein, the terms “a,” “an,” and “the” can refer to one or more unless specifically noted otherwise.

The term “or” is not to be construed as identifying mutually exclusive options. For example, the phrase “X contains A or B” means that X contains A and not B, X contains B and not A, or X contains both A and B.

As used herein, the terms “subject,” “individual,” and “patient” are used interchangeably. The use of these terms does not imply any kind of relationship to a medical professional, such as a physician.

As used herein, the term “biological sample” is used to refer to any fluid or tissue that can be isolated from an individual. For example, a biological sample may be whole blood, plasma, serum, other blood fraction, urine, cerebrospinal fluid, tissue homogenate, saliva, amniotic fluid, bile, mucus, peritoneal fluid, lymphatic fluid, perspiration, tissues, tissue homogenate, buccal swabs, chorionic villus samples, and the like.

As used herein, the term “binding assay” includes any assay that functions, at least in part, by binding (or non-covalently coupling) an analyte to some structure designed to couple to the analyte. The term “binding assay” includes hybridization assays, such as bead-based hybridization assays that are commonly used in genetic testing.

As used herein, the term “multiplex” refers to an assay that is capable of simultaneously testing for a plurality of different analytes.

Other terms are defined throughout the specification.

### Binding Assays, Binding Structures, and Target Analytes

In at least one aspect, the invention provides methods for validating a binding assay. Such methods include providing a binding assay having sets of binding structures. As used herein, the term “providing” is to be construed broadly. For example, a technician in a clinical laboratory can be said to “provide” the assay, for example, by preparing the sets of binding structures for contact with a negative control solution or a test solution.

The invention is not limited to any particular type of binding assay so long as the assay includes a plurality of binding structures that are adapted to couple to one or more target analytes. In some embodiments, the binding assay is a hybridization assay, wherein the target analytes are polynucleotides. As used herein, the term “binding structure” refers to a solid surface, e.g., a coated surface. In embodiments of the invention, the binding structures are adapted to couple (e.g., in a non-covalent manner) to one or more target analytes. In some embodiments, the binding structures are supported. The invention is not limited to any particular form of support. In some embodiments, the support is a solid sheet, such that various regions on the surface of the sheet are adapted to couple to one or more target analytes. Common examples of such technologies include the BEADARRAY systems sold by Illumina. In other embodiments, the solid support is a bead or a plurality of beads, where the surface of each bead is adapted to couple to one or more target analytes (i.e., typically one or two target analytes). In such embodiments, the beads have a diameter of 500 nm to 1000 mm, or 1 μm to 500 μm, or 1 μm to 100 μm, or 1 μm to 50 μm, or 2 μm to 20 μm, or 2 μm to 10 μm. In some such embodiments, the beads have a diameter of about 4.0 μm, or 4.5 μm, or 5.0 μm, or 5.5 μm, or 6.0 μm, or 6.5 μm. Common examples of such bead-based technologies include the beads used in the xMAP and xTAG systems sold by Luminex.

In some embodiments, the binding assay includes sets of binding structures. A set of binding structures refers to a collection of binding structures of different types, where each type of binding structure is adapted to couple to one or more (typically, one or two) target analytes. A set can include a plurality of different types of binding structures; thus, a set of binding structures permits coupling to (and ultimately, detection of) a plurality of different target analytes (e.g., often up to 100, 200 or 500 different target analytes). In embodiments where the binding structures lie on the surface of beads, a set of binding structures will generally include a plurality of beads that have each been adapted to couple to a particular analyte or a small number of particular analytes (e.g., 1-2 different analytes). The set can include a number of different types of binding structures. As used herein, the term “type,” when used in reference to a binding structure, refers to the manner in which the solid surface is adapted so as to bind to a particular target analyte or a small number of target analytes. A set can include at least 10, or at least 25, or at least 50, or at least 75, or at least 80, or at least 90, or at least 95, or at least 100, or at least 150, or at least 200, or more different types of binding structures. The set can also include multiple copies of binding structures of the same type (e.g., on different beads). For example, in some embodiments, the set includes at least 2 binding structures of the same type, or at least 5 binding structures of the same type, or at least 10 binding structures of the same type, or at least 25 binding structures of the same type, or at least 50 binding structures of the same type, or at least 75 binding structures of the same type, or at least 100 binding structures of the same type, or at least 150 binding structures of the same type. Thus, a set of binding structures can include 10,000 or more different binding structures.

Binding assays of the invention include at least two sets of binding structures. In some embodiments, the assay includes at least 3 sets of binding structures, or at least 5 sets of binding structures, or at least 10 sets of binding structures, or at least 25 sets of binding structures, or at least 50 sets of binding structures, or at least 75 sets of binding structures. In some embodiments, such as when multi-well plates are employed, the assay includes up to 6 sets of binding structures, or up to 24 sets of binding structures, or up to 96 sets of binding structures, or up to 384 sets of binding structures. In some embodiments, each set of the two or more sets is placed in a separate well of a multi-well plate. In some embodiments, every well in the plate contains a set of binding structures. In other embodiments, not every well does.

As noted above, each binding structure is adapted to couple to a target analyte or a small number of target analytes. The nature of the adapting will depend on the nature of the target analyte. The invention is not limited to any particular target analytes. In some embodiments, the target analytes are polynucleotides, oligonucleotides, polypeptides, oligopeptides, antibodies, antibody fragments, small-molecule organic compounds, metabolites of a small-molecule organic compounds, viruses, or pathogens. In some such embodiments, the target analytes include polynucleotides and oligonucleotides (i.e., a short nucleic acid polymers containing 100 or fewer nucleic acid units). In some embodiments, the target analytes include antibodies or antibody fragments.

Target-adapted binding structures are well known in the art. In some embodiments, the adapting of the binding structure involves affixing molecules (e.g., polypeptides chains, polynucleotides, etc.) to the solid surface, wherein these molecules bind selectively to certain target analytes. The xTAG technology of Luminex provides at least one example of how one would adapt a binding structure (e.g., on the surface of a bead) to couple to a particular target analyte. In some embodiments, the target analytes include polynucleotides. In such embodiments, the target analyte is a polynucleotide having a specific mutation that has been amplified (e.g., by allele-specific primer extension (ASPE)). In some embodiments, such mutations may by indicative of vulnerability to an inherited disease. In such cases, the binding structures contain oligonucleotides and/or polynucleotides on their surface. These oligonucleotides and/or polynucleotides (which contain a complementary sequence to at least a part of the sequence of the target polynucleotide, e.g., the part amplified by ASPE) are bound to the surface of the bead and can couple to specific target analytes (i.e., target polynucleotides). In this way, the binding structure is adapted to couple to a target analyte. Using standard methodologies, one of skill in the art may create target-adapted binding structures that are designed to couple to a wide array of target analytes.

In some embodiments, the target analytes include polynucleotides. In some such embodiments, at least some of the polynucleotides contain genetic mutations that are associated with certain inherited diseases. In some embodiments, these mutations have been amplified by ASPE. In this way, the target polynucleotide is indicative of an inherited disease or of a vulnerability to an inherited disease. The phrase “vulnerability to an inherited disease” does not imply that a subject shows symptoms of the disease. In some embodiments, the vulnerability refers to an increased likelihood that a subject may develop a certain inherited disease. In other embodiments, the vulnerability refers to an increased likelihood that a subject's biological offspring will develop a certain inherited disease. Thus, the “vulnerability” need not relate directly to the subject who contributed a sample. The invention is not limited to any particular inherited disease. In some embodiments, the inherited disease is familial hypercholesterolenemia, polycystic kidney disease, neurofibromatosis type 1, neurofibromatosis type 2, hereditary spherocytosis, Marfan syndrome, Huntington's disease, sickle cell anemia, cyctic fibrosis, lysosomal acid lipase (LAL) deficiency, Tay-Sachs disease, phenylketonuria, mucopolysaccharidoses, glycogen storage diseases, galactosemia, Duchenne muscular dystrophy, hemophilia, hereditary nonpolyposis colorectal cancer, hereditary multiple exostoses, Niemann-Pick disease, spinal muscular atrophy, Roberts syndrome, X-linked phosphatemic rickets, Rett syndrome, incontinentia pigmenti type 2, Aicardi syndrome, Klinefelter syndrome, Lesch-Nyhan syndrome, male pattern baldness, Turner syndrome, hypertrichosis pinnae, Leber's hereditary optic neuropathy, asthma, ciliopathies, cancers, cleft palate, diabetes, heart disease, hypertension, inflammatory bowel disease, mental retardation, mood disorder, obesity, refractive disorder, or infertility. In some such embodiments, the inherited disease is cyctic fibrosis. Other diseases may also be assessed using the methods, kits, systems, and computer readable media of the invention.

In some embodiments, at least some of the polynucleotides correspond to genetic mutations that are associated with sensitivity or resistance to a course of treatment, such as a drug-based treatment. In some such embodiments, the course of treatment relates to administration of various small molecule compounds, e.g., for the treatment of cancer, heart attack or stroke. Such small molecule compounds include, but are not limited to erlotinib and gefitinib, vemurafenib, clopidogrel, and the like.

In some embodiments, the target analytes include polypeptides or oligopeptides, including antibodies or antibody fragments. In some such embodiments, the peptides or oligopeptides are indicative of a likelihood or showing responsiveness or resistance to a course of treatment, such as a drug-based course of treatment. In some such embodiments, the course of treatment relates to administration of various monoclonal antibodies, e.g., for the treatment of cancer or autoimmune diseases. Such monoclonal antibodies include, but are not limited to, bevacizumab, trastuzumab, adalimumab, infliximab, rituximab, and the like.

In some embodiments, the target analytes include viruses or biomarkers indicative of a viral infection. In some embodiments, the target analytes include antibodies (or fragments thereof), such as antibodies or antibody fragments that are indicative of human allergic responses, e.g., human IgE antibodies, or are indicative of immuno-rejection during organ transplant, or are indicative of the efficacy of a vaccination protocol, or are antibodies related to cellular signaling. In some embodiments, the target analytes include biomarkers, such as biomarkers indicative of a disease or condition, e.g., an autoimmune disease. In some embodiments, the target analytes include viruses, bacteria, parasites. In such embodiments, samples may be obtained from human subjects, or from the environment. In some embodiments, the target analytes include polynucleotides or polynucleotides that are indicative of adverse drug reactions. The target analytes can also include biomarkers for various diseases, cytokines, chemokines, and growth factors. They can also include small molecules, such as hormones.

### Assay Performance and Use of Control Solutions

Methods of the invention include contacting at least two sets of binding structures with two control solutions. In some embodiments, the methods include contacting at least one additional set of binding structures with a sample that may contain or is believed to contain at least one target analyte. Such samples are referred to herein as “test samples” because these are the samples that are being tested to determine whether they contain any of the target analytes. In contrast the control solutions are expected not to contain any of the target analytes, or are not expected to contain any substantial amount of the target analytes. In this sense, the control solutions are adapted to be free, or at least substantially free, of some or all of the target analytes. In other words, they are negative control solutions. If the control solutions contain one or more of the target analytes (or at least a substantial amount of a target analyte), it is an indication that the assay may have become corrupted through contamination of some sort. In this sense, the negative control solutions provide an indication as to whether the results for any test samples should be trusted.

The invention is not limited to any particular protocol for preparing test samples. The nature of the test sample will vary depending on the nature of the target analytes to be measured. The preparation of test samples is well known in the art; thus those of skill in the art are able to design appropriate protocols for generating test samples for quantitative or qualitative measurement of any of the target analytes.

In some embodiments, the test samples are derived from a biological sample. As used herein, the term “biological sample” refers to any fluid or tissue that isolated from a living source, such as a plant or animal. In some embodiments, the biological sample is a sample derived from a human subject. In some such embodiments, a biological sample may be whole blood, plasma, serum, other blood fraction, urine, cerebrospinal fluid, tissue homogenate, saliva, amniotic fluid, bile, mucus, peritoneal fluid, lymphatic fluid, perspiration, tissues, tissue homogenate, buccal swabs, chorionic villus samples and the like. In other embodiments, the test samples are environmental samples, meaning that they are derived from environmental samples, such as lakes, streams, groundwater, air, soil, and the like. Extracting and isolating target analytes from biological and environmental samples is known in the art.

In some embodiments, the test sample includes target analytes that are polynucleotides. In some such embodiments, these polynucleotides contain regions of interest of human DNA or RNA. Such regions of interest can relate to particular mutations that are indicative of vulnerability to an inherited disease, or diagnosis of an inherited disease, such as cystic fibrosis. In some such embodiments, the human DNA is obtained from a human subject, for example, by withdrawing blood or by obtaining a mouthwash sample. Isolation of DNA from such biological samples is well known in the art, and test kits useful for such purposes are commercially available. Following isolation, the polynucleotide or at least relevant portions thereof (e.g., portions related to a gene of interest, such as the CFTR gene) are amplified by PCR. Using certain enzymes, one can degrade unused reagents left over from the PCR (e.g., primers and the like). In some embodiments, the amplified polynucleotides are further transformed by allele specific primer extension (ASPE). For example, the amplified polynucleotides can be mixed with short oligonucleotide sequences that are specific to certain features of interest (e.g., certain mutations, such as mutations that are indicative of a disease or a vulnerability thereto), which will bind to the amplified DNA of the subject, and be lengthened if the amplified DNA contains the features of interest (e.g., certain mutations). The short oligonucleotide sequences may also contain features that allow the polynucleotide to couple to a binding structure that is adapted to couple to analytes where the target-specific sequence is amplified. Such approaches produce a polynucleotide that includes a target-specific sequence. In some embodiments, a reporter (or a site to which to attach the reporter, e.g., biotin) is incorporated. Reporters are described in further detail below. A solution comprising these polynucleotides can be referred to as a “test solution.”

When a test solution comprising polynucleotides (e.g., polynucleotides transformed to contain an amplified target-specific sequence) contact a set of target-adapted binding structures, such polynucleotides can couple to binding structures that are adapted to couple to them. In embodiments where the polynucleotide includes a reporter (or a reporter attachment site), the contacting causes one or more reporters to couple indirectly (via the extended primer) to binding structures that are adapted to couple selectively to the target analyte. Thus, if a particular target analyte is present in the test sample, the binding structure adapted to couple to that target analyte will become labeled with a reporter (or reporter attachment site) once the test solution contacts the set of binding structures.

As noted above, control solutions, such as negative control solutions, can be used in combination with the test solutions. The invention provides methods that employ the use of at least two negative control solutions. In some embodiments, two negative control solutions are employed. In other embodiments, three or more can be employed. Such solutions should generally contain the same ingredients as the test solutions, except that the negative control solutions are expected to be free of the target analytes. In some embodiments, such as those where the target analytes include polynucleotides or oligonucleotides, the negative control solutions are a no template control (NTC). Methods of preparing NTCs are well known in the art. In some embodiments, the negative control solutions are all taken from the same control solution. In other embodiments, at least one of the negative control solutions is prepared separately.

In embodiments where two negative control solutions are used, each solution contacts a different set of binding structures (i.e. sets of beads). Thus, there will be one set of binding structures that are contacted with a first negative control solution, and a second set of binding structures that are contacted with a second negative control. In some embodiments, the first negative control solution and the second negative control solution are simply two separate aliquots from the same batch of negative control solution. In other embodiments, the two negative control solutions are from separate batches.

In some embodiments, positive controls are also used. The positive controls can be prepared in a manner similar to the preparation of the test solutions, except that the positive control is derived from a source known to possess the tested features. In some embodiments, a positive control is derived from a subject previously confirmed to possess one or more of the target analytes, possess certain concentrations (e.g., elevated concentrations or depressed concentrations) of one or more of the target analytes, or possess DNA, which, when amplified by PCR and transformed by ASPE, represents a target analyte. The positive control can also be prepared synthetically by techniques well known in the art. The method of designing and preparing the positive control will vary depending on the identity of the target analytes. Solutions derived from positive controls are referred to herein as “positive control solutions.”

As noted above, in various embodiments, a set of binding structures is contacted with either a negative control solution, a test solution (if present), or a positive control solution (if present). The invention is not limited to any particular way of contacting the binding structures with a solution. Such methods will vary depending on the manner in which a set of binding structures is provided. Instructions for contacting the binding structures will generally be supplied by the manufacturer, and will be standard depending on the configuration of the sets of binding structures. For example, when the set of binding structures is a set of non-supported beads, e.g., such as those available from Luminex, the beads can be incubated with the solution in a test tube or the well of a plate. Suitable incubation times may vary depending on the nature of the target analytes. In some embodiments, incubation times are about 5 minutes, or about 10 minutes, or about 15 minutes, or about 20 minutes, or about 25 minutes, or about 30 minutes, or about 35 minutes, or about 40 minutes, or about 45 minutes.

In embodiments where a reporter attachment site is incorporated into a polynucleotide analyte, a set of binding structures can be washed with a solution comprising a reporter following the contacting of the binding structures with a test solution or a control solution. In general, reporters are molecules that can emit a signal, e.g., emission of a fluorescent or chemiluminescent signal following irradiation with light of a suitable wavelength. The invention is not limited to any particular type of reporter. Suitable reporters, such as fluorescent reporters or chemiluminescent reporters, are well known in the art. In some embodiments, the reporter is streptavidin conjugated to R-phycoerythrin. In embodiments where biotin is incorporated into a tag, the biotin can bind to the R-phycoerythrin-streptavidin conjugate.

### Determining a Binding Signal for the Binding Structures

Methods of the invention include determining a binding signal at least for the two or more sets of binding structures that are contacted with negative control solution. In embodiments where reporters are incorporated into or are attached to the target analyte, the binding signal, in some such embodiments, is a signal emitted by a reporter that has become affixed to a binding structure via the coupling of the target analyte to the binding structure. In some embodiments, the binding signal is a chemiluminescent signal. In some embodiments, the signal is a fluorescent signal. In some such embodiments, the emission is induced by irradiating the reporter with light of a certain wavelength, e.g., by a laser. The fluorescent or chemiluminescent emissions are detected by standard detection systems known in the art. Any suitable detection system can be used. The system may vary depending on the identity of the reporter, as different reporters may emit light at different wavelengths. The phrase “determining a binding signal” also includes detecting the absence of a signal. In situations where the solution contains none of a particular target analyte, no target analyte should couple to the binding structures adapted to couple to that particular target analyte. Thus, such binding structures should not have any reporters attached to them, and should not emit, for example, a fluorescent signal when it is illuminated with a laser. Thus, in this instance, the detecting step involves detecting the absence of a fluorescent signal. Nonetheless, such a negative result still falls within the scope of what is meant by “determining a binding signal.”

Various means are used to identify each type of binding structure within the set of binding structures. In embodiments where the binding structures are affixed to a solid sheet, the identity of the type of binding structure can be determined by the position of the binding structure on the solid support, as the binding structures will not move around during the contacting steps. In embodiments where the binding structures are affixed to the surface of beads, the beads can be identified by some suitable characteristic of the bead, including size, color, identifying features on the bead surface, etc. In some such embodiments, different bead types are differentiated by color. For example, the beads can be filled with different combinations of dyes (e.g., fluorescent dyes). Thus, a second laser can be used to irradiate the dye-filled beads and the resulting signal is detected. Thus, in some embodiments, the bead may be illuminated by two different lasers, one to induce fluorescence or chemiluminescence of any reporters that may be present, and another to induce fluorescence of the dye solution that fills the beads. Each of these signals is detected by suitable detection means. This dual irradiation need not occur in any particular order or sequence. It can occur simultaneously, or in sequence, with one occurring before the other.

### Validation Determination

Methods of the invention include comparing the determined binding signals to a predetermined threshold and a predetermined limit for one or more of the target analytes, so as to determine whether the assay is validated. As used herein, the term “validated” refers to a determination that the assay should not be failed and the results derived from all test samples are valid. As noted above, the assays can be performed using a number of different sets of binding structures (or beads). At least two of these sets are contacted with negative control solutions. But, in some embodiments, one or more sets of binding structures is contacted with a test solution, such that the determination of a binding signal for such sets of binding structures gives an indication of whether a target analyte is present in the test solution. The use of two or more negative controls serves the purpose of identifying potential problems with the assay that should cause one to be skeptical about the results obtained for the test solutions (e.g., whether contamination has occurred). Depending on the results obtained from determining the binding signals for the binding structures contacted with control solutions, one will either pass the assay (i.e., determine that it is validated) or one will fail the assay (i.e., determine that it is invalidated).

In traditional validation protocols, one may use a single negative control. For that single control, the control solution is contacted with a set of binding structures and a binding signal may be determined for each binding structure. A predetermined threshold is set. If the binding signal for any of the binding structures (or the mean binding signal for all binding structures of the same type) exceeds the predetermined threshold, the assay is rejected. It was discovered that this leads to a high number of false invalidations.

In methods of the invention, at least two control solutions are used instead of a single control solution. Also, two predetermined cutoffs may be employed: a predetermined threshold and a predetermined limit, to which the determined binding signals are compared for the sets of beads that are contacted with the negative control solutions.

As used herein, the phrase “comparing the determined binding signals” encompasses a range of different types of comparison strategies. In some embodiments, each of the two or more sets of binding structures contacted with a negative control solution has only one binding structure for each type of binding structure in the set. For such embodiments, the comparing includes comparing a binding signal for each binding structure in each set against the predetermined threshold and the predetermined limit. But in other embodiments, at least one of the two or more sets of binding structures contacted with a negative control solution has at least two or more binding structures for at least some (if not all) types of binding structures in the set. For such embodiments, one can determine two or more binding signals associated with a single type of binding structure in the set. In some such embodiments, one can perform the comparing step using a representative value of the determined binding signals for all binding structures of the same type within a single set. Such a representative value is mathematically calculated from the measured values and is arrived at by any suitable statistical or mathematical means of identifying a single value for the binding signal that can be representative of the determined binding signals for some or all binding structures of the same type within a single set. This can include employing an arithmetic mean, a mode, a weighted mean, a median, and so forth. In some embodiments, one or more of the determined binding signals may not be used in calculating the representative value (e.g., because it is determined to be an outlier, etc.).

Thus, in some embodiments, for each type of binding structure in one of the two or more sets of binding structures contacted with a negative control solution, one obtains a single number that can be compared to the predetermined threshold and the predetermined limit for each target analyte. In embodiments where two sets of binding structures are contacted with two negative control solutions (i.e., one negative control solution for each set of binding structures), one obtains a single number that can be compared to the predetermined threshold and the predetermined limit for each target analyte for each type of binding structure in each of the two sets of binding structures. Each of these numbers then is compared to the predetermined threshold and the predetermined limit for each target analyte.

The invention is not limited to any particular way of establishing the predetermined limit and predetermined threshold. Methods of establishing a predetermined threshold are well known in the art, and are well within the ability of the person of skill in the relevant art. Establishing a suitable predetermined threshold depends on a number of factors, including, but not limited to, the identity of the target analyte, the type of binding structure, the means of adapting the binding structures to couple to a target analyte, the identity of the reporter, the means of generating a binding signal, and the means of determining the binding signal. In embodiments where the target analytes include polynucleotides, where the binding structures are affixed to free-standing beads of 5-6 μm in diameter, and where the reporter is streptavidin conjugated to R-phycoerythrin, which is coupled to biotin, the predetermined threshold is a measurement of fluorescent intensity. As is well known in the art, fluorescent intensity is generally calculated by converting a luminous flux measurement that is converted into a current, amplified, converted to a voltage, and then digitized. As can be appreciated, this fluorescent intensity can depend on various factors, including but not limited to, the wavelength of the fluorescent radiation, the detection means, the amplification means, the methods for digital conversion, etc. In embodiments using a standard flow cytometry setup, such as the Luminex 100/200 system, the predetermined threshold for a target analyte, can for example be at least 10 MFI (mean fluorescent intensity), or at least 20 MFI, or at least 25 MFI, or at least 35 MFI, or at least 40 MFI, and no more than 50 MFI, or no more than 100 MFI, or no more than 200 MFI, or no more than 500 MFI, or no more than 1000 MFI. The predetermined limit is higher in value than the predetermined threshold. In some embodiments, the predetermined limit is at least about 1.5 times, or at least about 2.0 times, or at least about 3.0 times, and no more than 4.0 times, or no more than 5.0 times, or no more than 6.0 times, or no more than 7.0 times, the MFI of the predetermined threshold value. In some embodiments, depending on the value of the predetermined threshold, the predetermined limit is at least 50 MFI, and no more than 250 MFI, or no more than 500 MFI, or no more than 700 MFI, or no more than 900 MFI, or no more than 900 MFI, or no more than 1000 MFI, or no more than 2500 MFI. In some embodiments, different predetermined thresholds and predetermined limits are used for one or more bead types. In some embodiments, the same predetermined threshold and predetermined limit is used for all bead types. In some embodiments, the predetermined limit is 2-7 times, or 3-6 times, or 4-5 times the value of the predetermined threshold.

As noted above, in some embodiments, for each type of binding structure in one of the two or more sets of binding structures contacted with a negative control solution, one obtains a single number that can be compared to the predetermined threshold and the predetermined limit for each target analyte. Thus, for such embodiments, for each type of binding structure, there are two or more values to be compared against the predetermined threshold and the predetermined limit (i.e., one value for each of the two or more sets of binding structures, which are contacted with the two or more negative control solutions). In embodiments where two sets of binding structures are contacted with two negative control solutions (i.e., one negative control solution for each set of binding structures), there are two values for each binding structure type to be compared against the predetermined threshold and the predetermined limit (i.e., one value for each of the two sets of binding structures, which are contacted with the two negative control solutions).

Table 1, below, summarizes the certain test results for determining whether a binding assay passes or fails. Table 1 shows the results for a single type of binding structure from two different sets of binding structures that are contacted with a negative control solution. The two sets are denominated by S1 and S2. V stands for the determined binding signal for that particular type of binding structure from the set of binding structures (or a representative value, e.g., average, of multiple determined binding signals). PT and PL represent the predetermined threshold and the predetermined limit, respectively, where the predetermined limit is higher in value than the predetermined threshold. The column labeled “2-Control” represents the result obtained (i.e., pass or fail) under methods of the invention. The column labeled “1-Control” represents the results that would be obtained, where an assay is deemed a failure if any V exceeds the predetermined threshold (i.e., using a single control). Table 1 contemplates that each set of binding structures contains one or more (e.g., up to 100, 200, 500 or more) types of binding structures, and that, for all other types of binding structures, V≦PT for both sets, S1 and S2. The X indicates the observed relationship of V to PT and PL.

TABLE 1

| | Bead | | | | | |
|---|---|---|---|---|---|---|
| | Set | V ≦ PT | PT < V ≦ PL | V > PL | 2-Control | 1-Control |
| A | S1 | X | | | Pass | Pass |
| | S2 | X | | | | |
| B | S1 | X | | | Pass | Fail |
| | S2 | | X | | | |
| C | S1 | | X | | Fail | Fail |
| | S2 | | X | | | |

In some such embodiments, two different types of binding structures can have a value for the binding signal (or a representative value of multiple determined binding signals) that is above the predetermined threshold, and all other types of binding structures have V≦PT for both sets, S1 and S2. In embodiments of the invention, some such scenarios result in a pass while others result in a fail. Table 2 shows those results, using the same terminology as Table 1. Different types of binding structures are indicated by T1 and T2. Thus S1,T1 represents a first type of binding structure from the first set, S1,T2, represents a second type of binding structure from the first set, S2,T1 represents a first type of binding structure from the second set, and so on.

TABLE 2

| | Bead | | | | | |
|---|---|---|---|---|---|---|
| | Set | V ≦ PT | PT < V ≦ PL | V > PL | 2-Control | 1-Control |
| D | S1, T1 | | X | | Pass | Fail |
| | S1, T2 | | X | | | |
| | S2, T1 | X | | | | |
| | S2, T2 | X | | | | |
| E | S1, T1 | X | | | Fail | Fail |
| | S1, T2 | | X | | | |
| | S2, T1 | | X | | | |
| | S2, T2 | X | | | | |

As shown in Table 3, if V>PL for any type of binding structure within either of the two sets, it is a fail.

TABLE 3

| | Bead | | | | | |
|---|---|---|---|---|---|---|
| | Set | V ≦ PT | PT < V ≦ PL | V > PL | 2-Control | 1-Control |
| F | S1 | X | | | Fail | Fail |
| | S2 | | | X | | |
| G | S1 | | | X | Fail | Fail |
| | S2 | X | | | | |

Also, if V>PT for any three types of binding structures within the two sets, collectively, then the assay is a fail (and is invalidated). The advantage of the present invention is illustrated by the scenarios identified as B in Table 1 and D in Table 2. These represent assays that would be invalidated under the single-control method, but that would be validated when the validation is performed according to embodiments of the invention.

### Systems for Validating Assays

In at least one aspect, the invention provides systems for validating a multiplex binding assay having two or more sets of binding structures wherein each set of binding structures comprises at least one target-adapted binding structure that is adapted to couple to a target analyte, the system comprising: (a) a station for contacting a first set of the two or more sets of binding structures with a first negative control solution; (b) a station for contacting a second set of the two or more sets of binding structures with a second negative control solution; (c) a station for determining a binding signal for the at least one target-adapted binding structure from the first set of two or more binding structures; (d) a station for determining a binding signal for the at least one target-adapted binding structure from the second set of two or more binding structures; and (e) a station for comparing each determined binding signal, or a representative value thereof, to a predetermined threshold and a predetermined limit to determine whether the binding assay is validated.

In another aspect, the invention provides systems for validating a multiplex binding assay having two or more sets of binding structures, wherein each set of binding structures comprises at least one target-adapted binding structure of a first type that is adapted to couple to a first target analyte, and at least one target-adapted binding structure of a second type that is adapted to couple to a second target analyte, the system comprising: (a) a station for contacting a first set of the two or more sets of binding structures with a first negative control solution; (b) a station for contacting a second set of the two or more sets of binding structures with a second negative control solution; (c) a station for determining a binding signal for the at least one target-adapted binding structure of the first type from the first set of two or more binding structures, and a binding signal for the at least one target-adapted binding structure of the second type from the first set of two or more binding structures; (d) a station for determining a binding signal for the at least one target-adapted binding structure of the first type from the second set of two or more binding structures, and a binding signal for the at least one target-adapted binding structure of the second type from the second set of two or more binding structures; and (e) a station for comparing each determined binding signal, or a representative value thereof, to a predetermined threshold and a predetermined limit to determine whether the binding assay is validated.

Such systems can include various embodiments and subembodiments analogous to those described above for methods of validating a binding assay.

These systems include various stations. As used herein, the term “station” is broadly defined and includes any suitable apparatus or collections of apparatuses suitable for carrying out the recited method. The stations need not be integrally connected or situated with respect to each other in any particular way. The invention includes any suitable arrangements of the stations with respect to each other. For example, the stations need not even be in the same room. But in some embodiments, the stations are connected to each other in an integral unit.

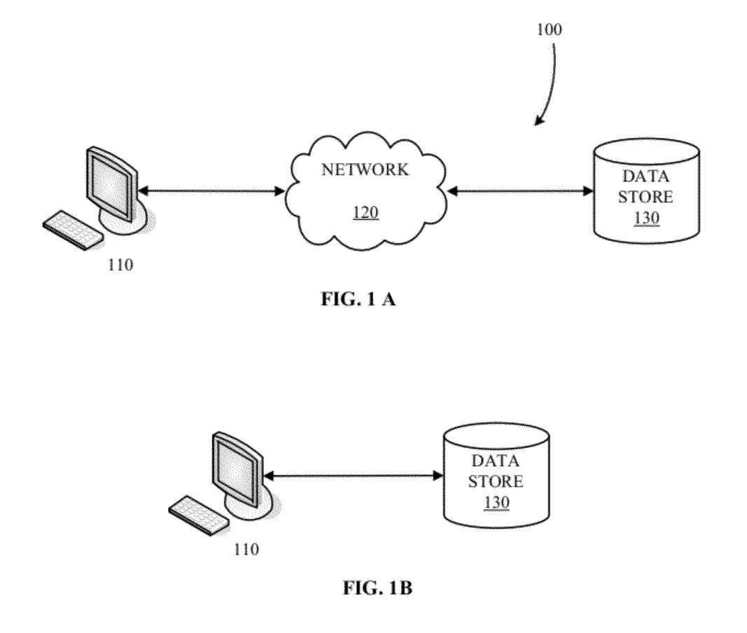

FIGS. 1A and 1B show embodiments of systems suitable for executing certain steps of the methods disclosed herein. For example, FIGS. 1A and 1B show diagrams depicting illustrative computing devices in illustrative computing environments according to some embodiments. The system 100 shown in FIG. 1A includes a computing device 110, a network 120, and a data store 130. The computing device 110 and the data store 130 are connected to the network 120. In this embodiment, the computing device 110 can communicate with the data store 130 through the network 120.

The system 100 shown in FIG. 1A includes a computing device 110. A suitable computing device for use with some embodiments may comprise any device capable of communicating with a network, such as network 120, or capable of sending or receiving information to or from another device, such as data store 130. A computing device can include an appropriate device operable to send and receive requests, messages, or information over an appropriate network. Examples of such suitable computing devices include personal computers, cell phones, handheld messaging devices, laptop computers, tablet computers, set-top boxes, personal data assistants (PDAs), servers, or any other suitable computing device. In some embodiments, the computing device 110 may be in communication with other computing devices directly or through network 120, or both. For example, in FIG. 1B, the computing device 110 is in direct communication with data store 130, such as via a point-to-point connection (e.g. a USB connection), an internal data bus (e.g. an internal Serial ATA connection) or external data bus (e.g. an external Serial ATA connection). In one embodiment, computer device 110 may comprise the data store 130. For example, in one embodiment, the data store 130 may comprise a hard drive that is a part of the computer device 110.

A computing device typically will include an operating system that provides executable program instructions for the general administration and operation of that computing device, and typically will include a computer-readable storage medium (e.g., a hard disk, random access memory, read only memory, etc.) storing instructions that, when executed by a processor of the server, allow the computing device to perform its intended functions. Suitable implementations for the operating system and general functionality of the computing device are known or commercially available, and are readily implemented by persons having ordinary skill in the art, particularly in light of the disclosure herein.

In the embodiment shown in FIG. 1A, the network 120 facilitates communications between the computing device 110 and the data store 130. The network 120 may be any suitable number or type of networks or links, including, but not limited to, a dial-in network, a local area network (LAN), wide area network (WAN), public switched telephone network (PSTN), the Internet, an intranet or any combination of hard-wired and/or wireless communication links. In one embodiment, the network 120 may be a single network. In other embodiments, the network 120 may comprise two or more networks. For example, the computing device 110 may be connected to a first network and the data store 130 may be connected to a second network and the first and the second network may be connected. In one embodiment, the network 120 may comprise the Internet. Components used for such a system can depend at least in part upon the type of network and/or environment selected. Protocols and components for communicating via such a network are well known and will not be discussed herein in detail. Communication over the network can be enabled by wired or wireless connections, and combinations thereof. Numerous other network configurations would be obvious to a person of ordinary skill in the art.

The system 100 shown in FIG. 1A includes a data store 130. The data store 130 can include several separate data tables, databases, or other data storage mechanisms and media for storing data relating to a particular aspect. It should be understood that there can be many other aspects that may need to be stored in the data store, such as to access right information, which can be stored in any appropriate mechanism or mechanisms in the data store 130. The data store 130 may be operable to receive instructions from the computing device 110 and obtain, update, or otherwise process data in response thereto.

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (“SAN”) familiar to those skilled in the art. Similarly, any necessary files for performing the functions attributed to the computers, servers, or other network devices may be stored locally and/or remotely, as appropriate. Where a system includes computing devices, each such device can include hardware elements that may be electrically coupled via a bus, the elements including, for example, at least one central processing unit (CPU), at least one input device (e.g., a mouse, keyboard, controller, touch screen, or keypad), and at least one output device (e.g., a display device, printer, or speaker). Such a system may also include one or more storage devices, such as disk drives, optical storage devices, and solid-state storage devices such as random access memory (“RAM”) or read-only memory (“ROM”), as well as removable media devices, memory cards, flash cards, etc.

Such devices also can include a computer-readable storage media reader, a communications device (e.g., a modem, a network card (wireless or wired), an infrared communication device, etc.), and working memory as described above. The computer-readable storage media reader can be connected with, or configured to receive, a computer-readable storage medium, representing remote, local, fixed, and/or removable storage devices as well as storage media for temporarily and/or more permanently containing, storing, transmitting, and retrieving computer-readable information. The system and various devices also typically will include a number of software applications, modules, services, or other elements located within at least one working memory device, including an operating system and application programs, such as a client application or Web browser. It should be appreciated that alternate embodiments may have numerous variations from that described above. For example, customized hardware might also be used and/or particular elements might be implemented in hardware, software (including portable software, such as applets), or both. Further, connection to other computing devices such as network input/output devices may be employed.

Storage media and computer readable media for containing code, or portions of code, can include any appropriate media known or used in the art, including storage media and communication media, such as but not limited to volatile and non-volatile, removable and non-removable media implemented in any method or technology for storage and/or transmission of information such as computer readable instructions, data structures, program modules, or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices, or any other medium which can be used to store the desired information and which can be accessed by the a system device. Based on the disclosure and teachings provided herein, a person of ordinary skill in the art will appreciate other ways and/or methods to implement the various embodiments.

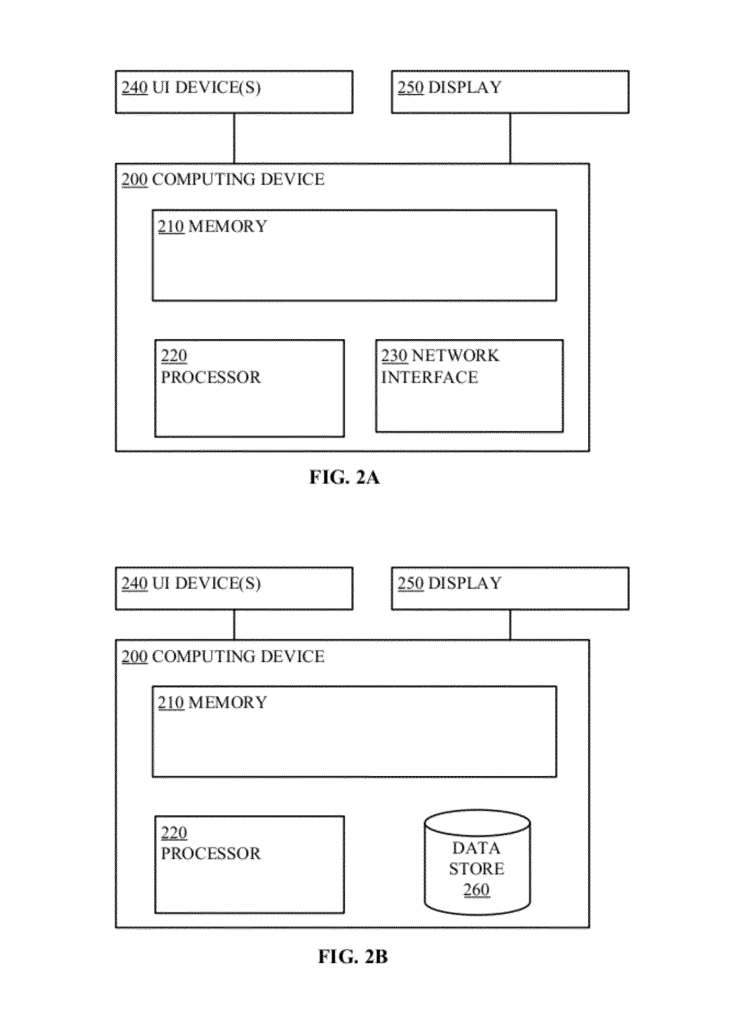

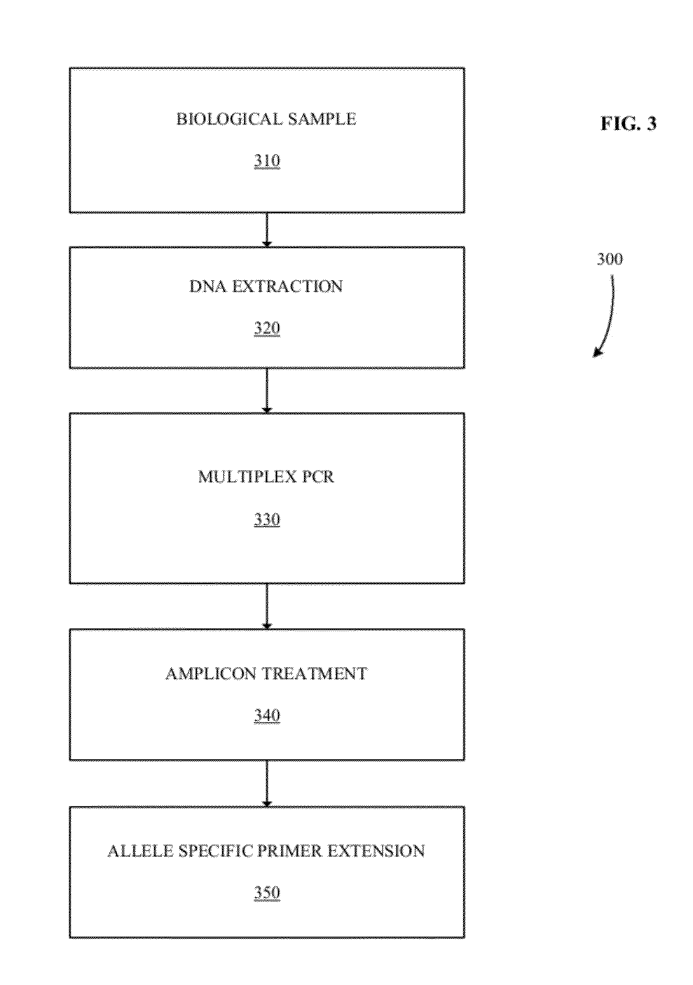

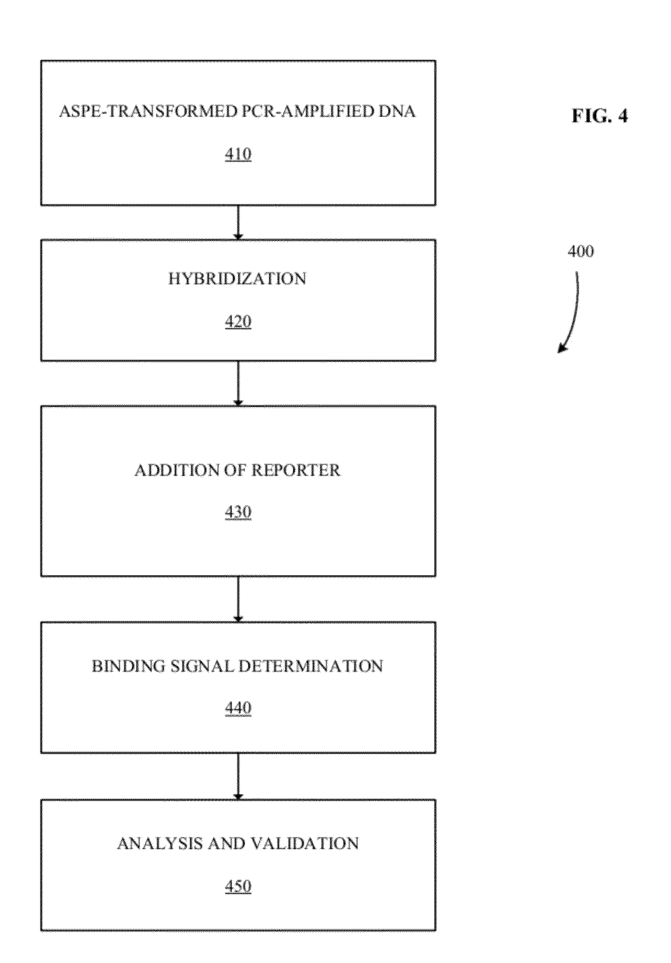

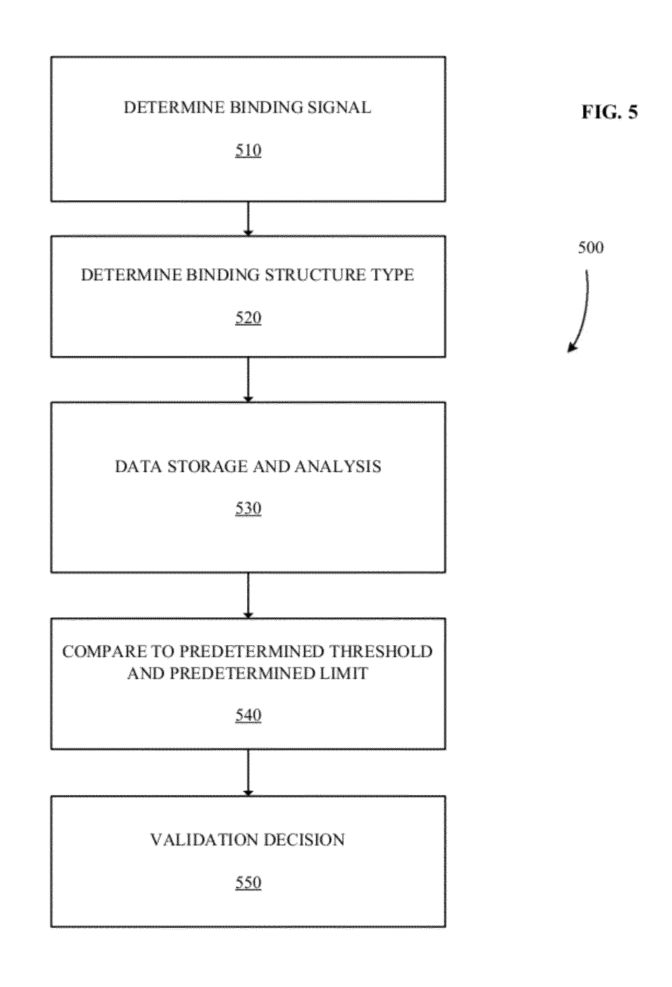

FIGS. 2A and 2B show block diagrams depicting illustrative computing devices according to various embodiments. According to the embodiment shown in FIG. 2A, the computing device 200 comprises a computer-readable medium such as memory 210 coupled to a processor 220 that is configured to execute computer-executable program instructions (or program code) and/or to access information stored in memory 210. A computer-readable medium may comprise, but is not limited to, an electronic, optical, magnetic, or other storage device capable of providing a processor with computer-readable instructions. Other examples include, but are not limited to, a floppy disk, CD-ROM, DVD, magnetic disk, memory chip, ROM, RAM, SRAM, DRAM, content-addressable memory (“CAM”), DDR, flash memory such as NAND flash or NOR flash, an ASIC, a configured processor, optical storage, magnetic tape or other magnetic storage, or any other medium from which a computer processor can read instructions. In one embodiment, the computing device 200 may comprise a single type of computer-readable medium such as random access memory (RAM). In other embodiments, the computing device 200 may comprise two or more types of computer-readable medium such as random access memory (RAM), a disk drive, and cache. The computing device 200 may be in communication with one or more external computer-readable mediums such as an external hard disk drive or an external DVD drive.

As discussed above, the embodiment shown in FIG. 2A comprises a processor 220 which is configured to execute computer-executable program instructions and/or to access information stored in memory 210. The instructions may comprise processor-specific instructions generated by a compiler and/or an interpreter from code written in any suitable computer-programming language including, for example, C, C++, C#, Visual Basic, Java, Python, Perl, JavaScript, and ActionScript (Adobe Systems, Mountain View, Calif.). In an embodiment, the computing device 200 comprises a single processor 220. In other embodiments, the device 200 comprises two or more processors. Such processors may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

The computing device 200 as shown in FIG. 2A comprises a network interface 230. In some embodiments, the network interface 230 is configured for communicating via wired or wireless communication links. For example, the network interface 230 may allow for communication over networks via Ethernet, IEEE 802.11 (Wi-Fi), 802.16 (Wi-Max), Bluetooth, infrared, etc. As another example, network interface 230 may allow for communication over networks such as CDMA, GSM, UMTS, or other cellular communication networks. In some embodiments, the network interface may allow for point-to-point connections with another device, such as via the Universal Serial Bus (USB), 1394 FireWire, serial or parallel connections, or similar interfaces. Some embodiments of suitable computing devices may comprise two or more network interfaces for communication over one or more networks. In some embodiments, such as the embodiment shown in FIG. 2B, the computing device may include a data store 260 in addition to or in place of a network interface.

Some embodiments of suitable computing devices may comprise or be in communication with a number of external or internal devices such as a mouse, a CD-ROM, DVD, a keyboard, a display, audio speakers, one or more microphones, or any other input or output devices. For example, the computing device 200 shown in FIG. 2A is in communication with various user interface devices 240 and a display 250. Display 250 may use any suitable technology including, but not limited to, LCD, LED, CRT, and the like.

In various embodiments, suitable computing devices may be a server, a desktop computer, a personal computing device, a mobile device, a tablet, a mobile phone, or any other type of electronic devices appropriate for providing one or more of the features described herein. In at least one aspect, the invention provides systems for carrying out the analysis described above. Thus, in some embodiments, the present invention comprises a computer-readable medium on which is encoded programming code for the generalized ridge regression methods described herein. Also in some embodiments, such as described above with respect to FIGS. 1 and 2, the invention comprises a system comprising a processor in communication with a computer-readable medium, the processor configured to perform the generalized ridge regression methods described herein. Suitable processors and computer-readable media for various embodiments of the present invention are described in greater detail above.

In at least one aspect, the invention provides computer readable media for validating a multiplex binding assay, the computer readable medium comprising: (a) program code for determining a binding signal for a target-adapted binding structure, where the target-adapted binding structure is a binding structure that is adapted to couple to a target analyte and subsequently contacted with a first negative control solution; (b) program code for determining a binding signal for a target-adapted binding structure, where the target-adapted binding structure is a binding structure that is adapted to couple to a target analyte and subsequently contacted with a second negative control solution; (c) program code for comparing each determined binding signal, or a representative value thereof, to a predetermined threshold and a predetermined limit; and (d) program code for determining whether to determine whether the multiplex binding assay is validated.

In another aspect, the invention provides computer readable medium for validating a multiplex binding assay, the computer readable medium comprising: (a) program code for determining a binding signal for a target-adapted binding structure of a first type, where the target-adapted binding structure is a binding structure that is adapted to couple to a first target analyte and subsequently contacted with a first negative control solution; (b) program code for determining a binding signal for a target-adapted binding structure of a second type, where the target-adapted binding structure is a binding structure that is adapted to couple to a second target analyte and subsequently contacted with a first negative control solution; (c) program code for determining a binding signal for a target-adapted binding structure of a first type, where the target-adapted binding structure is a binding structure that is adapted to couple to a first target analyte and subsequently contacted with a second negative control solution; (d) program code for determining a binding signal for a target-adapted binding structure of a second type, where the target-adapted binding structure is a binding structure that is adapted to couple to a second target analyte and subsequently contacted with a second negative control solution; (e) program code for comparing each determined binding signal, or a representative value thereof, to a predetermined threshold and a predetermined limit; and (f) program code for determining whether the multiplex binding assay is validated.

Such computer readable media can include various embodiments and subembodiments analogous to those described above for methods of validating a binding assay, including other embodiments of methods described throughout this specification. For example, after the beads of each type of binding structure have been contacted by the two negative control solutions, flow cytometry can be used to arrange a set of beads in a line, and to direct each bead past a station for determining the identity of the bead (and thereby the identity of the binding structure on the surface of the bead) and a station for determining a binding signal from the bead. Each of these stations can include a laser for inducing fluorescence and a detector for detecting the resulting fluorescence. Data are generated from each of the detections. These data can be collected, amplified, and converted to digital form, and then may be compiled and/or transformed, if necessary, using any standard spreadsheet software such as Microsoft Excel, FoxPro, Lotus, or the like, or standard statistical algorithms. In an embodiment, the data are entered into the system for each bead and/or stored in memory. Alternatively, data from previous beads are stored in the computer memory and used as required.

At each point in the analysis, the user may input instructions via a keyboard, floppy disk, flash memory, remote access (e.g., via the Internet), or other access means. The user may enter instructions including options for the run, how reports should be printed out, and the like. Also, at each step in the analysis, the data may be stored in the computer using a storage device common in the art such as disks, drives or memory. As is understood in the art, the processor and I/O controller are required for multiple aspects of computer function. Also, in an embodiment, there may be more than one processor, wherein each processor is configured to perform some portion of a method disclosed by this specification and such processors are in communication with each other or another processor.

The data may also be processed to remove noise. In some cases, the user, via the keyboard, floppy disk, or remote access, may want to input variables or constraints for the analysis, as for example, the threshold for determining noise or for providing filtering algorithms or parameters.

It should be understood that the foregoing relates to certain embodiments of the invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein may suggest themselves to those skilled in the art without departing from the spirit of the present invention and/or the scope the appended claims.

### EXAMPLES

The following Examples illustrate certain embodiments of the invention. The Examples are not intended to serve as a source of limitations to be imposed on the claims. The Examples merely illustrate embodiments that fall within the scope of certain aspects of the invention.

### Example 1

### Multiplex Validation of Cystic Fibrosis Assay

Peripheral blood or mouthwash samples are received from human subjects. Genomic DNA is extracted from peripheral blood lymphocytes or buccal cells (from mouthwash samples) using the QIAmp 96 DNA Blood Kit (Qiagen). Minor modifications are made to reagent volumes, as needed. Regions of the cystic fibrosis transmembrane conductance regulator (CFTR) gene are amplified by PCR and subjected to multiplex allele-specific primer extension to generate polynucleotides having certain target-specific sequences amplified. Such target-specific sequences can relate to specific mutations in the CFTR gene that are useful in diagnosing cystic fibrosis or that are indicative of a vulnerability to cystic fibrosis. Such target-specific sequences can also relate to the corresponding regions of the wild type polynucleotide. Biotin is incorporated into the polynucleotides. To the test solution containing the amplified biotin-containing polynucleotides is added a solution containing streptavidin conjugated to R-phycoerythrin. The samples are then added to a well in 96-well plate. To two wells in the plate are negative control wells; to those wells is added a no template control (NTC) as a negative control, which has been prepared in the same way as the test samples but no subject DNA is incorporated. To one or more of the other wells in the plate, test solution is added. Each well in the plate contains a set of Luminex xMAP beads (bead array). Each bead has a surface that is adapted to hybridize to a polynucleotide in which a certain target-specific sequence has been amplified. The target specific sequence also includes sequence designed to hybridize to the binding structure. In some instances, the beads are adapted to hybridize to two different types of polynucleotides, in each of which a different target-specific sequence has been amplified. Each set of binding structures contains 100 beads of each type of binding structure, for beads having 100 different types of binding structures. After the sets of beads are incubated in the test solution or the negative control solution, each set is analyzed by the Luminex 100 to determine a fluorescent binding signal (in MFI) for each bead.

For each of the two sets that were incubated in the negative control solution, a mean binding signal is calculated for all of the beads of the same type within each of the two sets. Thus, for a bead having each type of binding structure in each set, one obtains a mean binding signal that represents the arithmetic mean of 100 readings. Each of these values is compared to a predetermined threshold of 200 MFI and a predetermined limit of 900 MFI. The tests shown in Tables 1-3 and in the associated text were used to determine whether the results for the test solutions (in the other wells in the plate) should be used (i.e., whether the assay passes and is therefore validated, or whether the assay fails and is therefore invalidated).

### Example 2

### Saving of Assays

Tables 1-3 show a comparison of pass/fail criteria for a two-control validation protocol and a one-control validation protocol (Comp.). During an 8-month period, an assay substantially identical to that described above was performed for 1236 96-well plates, where each plate contained two wells devoted to negative controls. The use of the two-control protocol resulted in the rescue of 15 plates that would have been falsely invalidated under a one-control protocol. This represented a saving of 1364 patient samples that would have had to have been collected anew if not for the use of the two-control validation protocol.

We claim:

1. A method for validating a multiplex binding assay, the method comprising:
(a) providing a binding assay having two or more sets of binding structures, wherein each set of binding structures comprises at least one target-adapted binding structure that is adapted to couple to a target analyte;
(b1) contacting a first set of the two or more sets of binding structures with a first negative control solution; and then (b2) determining a binding signal for the at least one target-adapted binding structure from the first set of two or more binding structures;
(c1) contacting a second set of the two or more sets of binding structures with a second negative control solution; and then (c2) determining a binding signal for the at least one target-adapted binding structure from the second set of two or more binding structures; and
(d) comparing each determined binding signal, or a value representative thereof, to a predetermined threshold and a predetermined limit to determine whether the binding assay is validated.

2. The method of claim 1, wherein each binding structure is on the outer surface of a bead.

3. The method of claim 2, wherein each bead has a diameter of 1 μm to 500 μm.

4. The method of claim 1, wherein the target analyte is a polynucleotide, an oligonucleotide, a polypeptide, an oligopeptide, an antibody, an antibody fragment, a small-molecule organic compound, a metabolite of a small-molecule organic compound, a virus, or a pathogen.

5. The method of claim 4, wherein the target analyte is a polynucleotide.

6. The method of claim 5, wherein the analyte is a polynucleotide comprising a target-specific nucleotide sequence that includes a mutation that is indicative of an inherited disease or indicative of a vulnerability to an inherited disease.

7. The method of claim 6, where the inherited disease is familial hypercholesterolenemia, polycystic kidney disease, neurofibromatosis type 1, neurofibromatosis type 2, hereditary spherocytosis, Marfan syndrome, Huntington's disease, sickle cell anemia, cystic fibrosis, lysosomal acid lipase (LAL) deficiency, Tay-Sachs disease, phenylketonuria, mucopolysaccharidoses, glycogen storage diseases, galactosemia, Duchenne muscular dystrophy, hemophilia, hereditary nonpolyposis colorectal cancer, hereditary multiple exostoses, Niemann-Pick disease, spinal muscular atrophy, Roberts syndrome, X-linked phosphatemic rickets, Rett syndrome, incontinentia pigmenti type 2, Aicardi syndrome, Klinefelter syndrome, Lesch-Nyhan syndrome, male pattern baldness, Turner syndrome, hypertrichosis pinnae, Leber's hereditary optic neuropathy, asthma, ciliopathies, cancers, cleft palate, diabetes, heart disease, hypertension, inflammatory bowel disease, mental retardation, mood disorder, obesity, refractive disorder, or infertility.

8. The method of claim 4, where the target analyte is a polynucleotide comprising a PCR-amplified region of the DNA of a human subject, where the PCR-amplified region includes one or more mutations that are indicative of an inherited disease or indicative of a vulnerability to an inherited disease.

9. The method of claim 8, where the inherited disease is familial hypercholesterolenemia, polycystic kidney disease, neurofibromatosis type 1, neurofibromatosis type 2, hereditary spherocytosis, Marfan syndrome, Huntington's disease, sickle cell anemia, cyctic fibrosis, lysosomal acid lipase (LAL) deficiency, Tay-Sachs disease, phenylketonuria, mucopolysaccharidoses, glycogen storage diseases, galactosemia, Duchenne muscular dystrophy, hemophilia, hereditary nonpolyposis colorectal cancer, hereditary multiple exostoses, Niemann-Pick disease, spinal muscular atrophy, Roberts syndrome, X-linked phosphatemic rickets, Rett syndrome, incontinentia pigmenti type 2, Aicardi syndrome, Klinefelter syndrome, Lesch-Nyhan syndrome, male pattern baldness, Turner syndrome, hypertrichosis pinnae, Leber's hereditary optic neuropathy, asthma, ciliopathies, cancers, cleft palate, diabetes, heart disease, hypertension, inflammatory bowel disease, mental retardation, mood disorder, obesity, refractive disorder, or infertility.

10. The method of claim 1, wherein the target analyte is indicative of a likelihood of resistance to a drug-based therapy.

11. A method for validating a multiplex binding assay, the method comprising:
(a) providing a binding assay having two or more sets of binding structures, wherein each set of binding structures comprises at least one target-adapted binding structure of a first type that is adapted to couple to a first target analyte, and at least one target-adapted binding structure of a second type that is adapted to couple to a second target analyte;
(b1) contacting a first set of the two or more sets of binding structures with a first negative control solution; and then (b2) determining a binding signal for the at least one target-adapted binding structure of the first type from the first set of two or more binding structures, and a binding signal for the at least one target-adapted binding structure of the second type from the first set of two or more binding structures;
(c1) contacting a second set of the two or more sets of binding structures with a second negative control solution; and then (b2) determining a binding signal for the at least one target-adapted binding structure of the first type from the second set of two or more binding structures, and a binding signal for the at least one target-adapted binding structure of the second type from the second set of two or more binding structures; and
(d) comparing each determined binding signal, or a value representative thereof, to a predetermined threshold and a predetermined limit to determine whether the binding assay is validated.

12. The method of claim 11, wherein each binding structure is on the outer surface of a bead.

13. The method of claim 12, wherein each bead has a diameter of 1 μm to 500 μm.

14. The method of claim 11, wherein each of the first target analyte and the second target analyte is a polynucleotide, an oligonucleotide, a polypeptide, an oligopeptide, an antibody, an antibody fragment, a small-molecule organic compound, a metabolite of a small-molecule organic compound, a virus, or a pathogen.

15. The method of claim 14, wherein each of the first target analyte and the second target analyte is a polynucleotide.

16. The method of claim 15, wherein each of the first target analyte and the second target analyte is a polynucleotide comprising a target-specific nucleotide sequence that includes a mutation that is indicative of an inherited disease or indicative of a vulnerability to an inherited disease.

17. The method of claim 16, where the inherited disease is familial hypercholesterolenemia, polycystic kidney disease, neurofibromatosis type 1, neurofibromatosis type 2, hereditary spherocytosis, Marfan syndrome, Huntington's disease, sickle cell anemia, cyctic fibrosis, lysosomal acid lipase (LAL) deficiency, Tay-Sachs disease, phenylketonuria, mucopolysaccharidoses, glycogen storage diseases, galactosemia, Duchenne muscular dystrophy, hemophilia, hereditary nonpolyposis colorectal cancer, hereditary multiple exostoses, Niemann-Pick disease, spinal muscular atrophy, Roberts syndrome, X-linked phosphatemic rickets, Rett syndrome, incontinentia pigmenti type 2, Aicardi syndrome, Klinefelter syndrome, Lesch-Nyhan syndrome, male pattern baldness, Turner syndrome, hypertrichosis pinnae, Leber's hereditary optic neuropathy, asthma, ciliopathies, cancers, cleft palate, diabetes, heart disease, hypertension, inflammatory bowel disease, mental retardation, mood disorder, obesity, refractive disorder, or infertility.

18. The method of claim 14, where each of the first target analyte and the second target analyte is a polynucleotide comprising a PCR-amplified region of the DNA of a human subject, where the PCR-amplified region includes one or more mutations that are indicative of an inherited disease or indicative of a vulnerability to an inherited disease.

19. The method of claim 18, where the inherited disease is familial hypercholesterolenemia, polycystic kidney disease, neurofibromatosis type 1, neurofibromatosis type 2, hereditary spherocytosis, Marfan syndrome, Huntington's disease, sickle cell anemia, cyctic fibrosis, lysosomal acid lipase (LAL) deficiency, Tay-Sachs disease, phenylketonuria, mucopolysaccharidoses, glycogen storage diseases, galactosemia, Duchenne muscular dystrophy, hemophilia, hereditary nonpolyposis colorectal cancer, hereditary multiple exostoses, Niemann-Pick disease, spinal muscular atrophy, Roberts syndrome, X-linked phosphatemic rickets, Rett syndrome, incontinentia pigmenti type 2, Aicardi syndrome, Klinefelter syndrome, Lesch-Nyhan syndrome, male pattern baldness, Turner syndrome, hypertrichosis pinnae, Leber's hereditary optic neuropathy, asthma, ciliopathies, cancers, cleft palate, diabetes, heart disease, hypertension, inflammatory bowel disease, mental retardation, mood disorder, obesity, refractive disorder, or infertility.

20. The method of claim 11, wherein each of the first target analyte or the second target analyte is indicative of a likelihood of resistance to a drug-based therapy.

21. A system for validating a multiplex binding assay having two or more sets of binding structures wherein each set of binding structures comprises at least one target-adapted binding structure that is adapted to couple to a target analyte, the system comprising:
(a) a station for contacting a first set of the two or more sets of binding structures with a first negative control solution;
(b) a station for contacting a second set of the two or more sets of binding structures with a second negative control solution;
(c) a station for determining a binding signal for the at least one target-adapted binding structure from the first set of two or more binding structures;
(d) a station for determining a binding signal for the at least one target-adapted binding structure from the second set of two or more binding structures; and
(e) a station for comparing each determined binding signal, or a value representative thereof, to a predetermined threshold and a predetermined limit to determine whether the binding assay is validated.

22. A system for validating a multiplex binding assay having two or more sets of binding structures, wherein each set of binding structures comprises at least one target-adapted binding structure of a first type that is adapted to couple to a first target analyte, and at least one target-adapted binding structure of a second type that is adapted to couple to a second target analyte, the system comprising:
(a) a station for contacting a first set of the two or more sets of binding structures with a first negative control solution;
(b) a station for contacting a second set of the two or more sets of binding structures with a second negative control solution;
(c) a station for determining a binding signal for the at least one target-adapted binding structure of the first type from the first set of two or more binding structures, and a binding signal for the at least one target-adapted binding structure of the second type from the first set of two or more binding structures;
(d) a station for determining a binding signal for the at least one target-adapted binding structure of the first type from the second set of two or more binding structures, and a binding signal for the at least one target-adapted binding structure of the second type from the second set of two or more binding structures; and
(c) a station for comparing each determined binding signal, or a representative value thereof, to a predetermined threshold and a predetermined limit to determine whether the binding assay is validated.

23. A computer readable medium for validating a multiplex binding assay, the computer readable medium comprising:
(a) program code for determining a binding signal for a target-adapted binding structure, where the target-adapted binding structure is a binding structure that is adapted to couple to a target analyte and subsequently contacted with a first negative control solution;
(b) program code for determining a binding signal for a target-adapted binding structure, where the target-adapted binding structure is a binding structure that is adapted to couple to a target analyte and subsequently contacted with a second negative control solution;
(c) program code for comparing each determined binding signal, or a value representative thereof, to a predetermined threshold and a predetermined limit; and
(d) program code for determining whether to determine whether the multiplex binding assay is validated.

24. A computer readable medium for validating a multiplex binding assay, the computer readable medium comprising:
(a) program code for determining a binding signal for a target-adapted binding structure of a first type, where the target-adapted binding structure is a binding structure that is adapted to couple to a first target analyte and subsequently contacted with a first negative control solution;
(b) program code for determining a binding signal for a target-adapted binding structure of a second type, where the target-adapted binding structure is a binding structure that is adapted to couple to a second target analyte and subsequently contacted with a first negative control solution;
(c) program code for determining a binding signal for a target-adapted binding structure of a first type, where the target-adapted binding structure is a binding structure that is adapted to couple to a first target analyte and subsequently contacted with a second negative control solution;
(d) program code for determining a binding signal for a target-adapted binding structure of a second type, where the target-adapted binding structure is a binding structure that is adapted to couple to a second target analyte and subsequently contacted with a second negative control solution;
(e) program code for comparing each determined binding signal, or a value representative thereof, to a predetermined threshold and a predetermined limit; and
(f) program code for determining whether the multiplex binding assay is validated.
